SEQUENCE LISTING (1 sequence)

SEQ ID NO: 1 - RNA, 15092 nt, synthetic construct
  misc_feature 1..15092: note = PRRS-1 virus isolate 96V198 - passage 49
  source 1..15092: mol_type = other RNA
atgatgtgtagggtattccccctacatacacgacacttctagtgtttgtgtgccttggaggcgtgggtatagccccgccccacctcttggcccctgttctagcccaacaggtatccttctctctcggggcgagcgcgccgcctgctgctcccttgcagcaggaaggacctcccgagtatttccggagagcacctgctttacgggatctccaccctttaaccatgtctgggacgttctcccggtgcatgtgcaccccggctgcccgggtattttggagcgccggccaagtctattgcacacggtgtctcagtgcacggtctcttctctctccggagcttcaggacactgacctcgctgcaattggcttgttttacaagcctaaagacaagcttcactggaaagtccctatcggcatccctcaggtggagtgtactccgtccgggtgctgttggctctcagccatcttccccttggcgcgcatgacctccggcaatcacaactttctccaacgacttgtgaaggttgctgatgttttgtaccgtgacggctgcttggcacctcgacaccttcgcgaactccaagtttacgagcgcggctgcaactggtacccgatcacggggcccgtgcccgggatgggtttgtacgcgaactccatgcacgtgtccgaccaaccgttccctggtgccgcttatgtgttaacgaactcacctctgcctcagcaggcttgtcgacagccgttttgtccatttgaggaggttcattccgacgtatacaggtggaagaaattcgtgatttttgtggattcctcttctgacggccgatctcgcatgatgtggacaccaggatccgacgactcggctgccttagaagtattgccacctgaactagaatgtcgagtcgaaatcctcgttcggagttttcctgcccaccaccctgtcgacatcaccaactgggagctcactgagtcccctgagcacggtttttccttcagcacgtctcattcttgtggttaccttgcccaagaccctgacgtgtttgatggtaagtgttggctttcttgctttttgggcctgccgaccgaagtgtggcgtcatgaggagcatctagctaacgccttcggttaccaaaccaagtggggcgtgcatggtaagtacctccagcgcaggcttcaggtccgcggcatgcgtgctgtagttgatcctgatggtcccatccacgttgaagcgctgtcttgcccccagtcttggatcaggcacctgactctaaatgatgacgtcaccccaggatttgttcgcctaacatcccttcgcattgagccgaacacagagcctactactttccggatctttcggtttggagcgcataagtggtatggcgctgccggcaaacgggctcgtgctaagcgtgccgctaaaggtgagaagaattcagctcccacccccaaggtcgccctgccggtccccacctatggaattaccacctactctccaccgacagacgggtcttgtggttggcatgtccttgccgccataatgaaccggatgataaatggtgacttcacgtcccctctgactccgtacaacagaccagaggatgattgggcttctgattatgatcttgctcaggcaatccaatgtctacaactgcctgctaccgtggtccggagtcgcgcctgtcccaacgccaagtacctcataaaactcaacggagtccactgggaggtagaggtgaggtcaggaatggctcctcgctctctttctcgtgaatgtgtggttggcgtttgctctgaaggttgtgttgcaccgccttatccagcagacgggctaccagaacgagcactcgaggccttggcgtctgcttacaggttaccctctgattgtgttagctctggcattgctgactttcttgctaacccacctcctcaggaattctggaccctcgacaaaatgttgacctccccgtcaccagagcggtccggcttctctagtttgtataaattactattagaggttgttccgcaaaaatgcggtgccacggaaggggctttcgtctatgctgttgagagaatgttgaaggattgtccgagctccaaacaggccatggcccttctggcaaaaattaaagttccatcctcaaaggccccgtctgtgtcccttgacgagtgtttccctacggatgtttcagccgacttcgagccagcatctcaggaaaggccccaaagttccggtgctgctgttgtcctgtgttcaccggaagtaaaagagttcgaagaagcagccccagaagaagttcaagagagtggccataaggtcgcccgctctgcatttgttgccgagggtcctaacaatgaacaggtaccgatggctgccggcgagcaactgaagcccggtggtcgcgttttggcggtcgggaatgctcatgaaggtgttctggcctcaactagttcgaccaacctgataggcgggaacttccccccttcagactccatgaaagagagcatgctcaatagctgggaagacgaaccactggatttgtcccaaccggcaccagctgttacaatgacccttgtgagagagcaaacacccgacaacctgggtcctgatgccggtgccttccccgtcaccgttcgaaaatttgtcccgacagggcctacactccgtcatgttgagcactgcggcacggagtcaggcgacagcagttcgcccttggatctgtcttatgcgcaaaccctggaccagcctttaaatctatccttggccgcttggccagtgagggccaccgcgtctgaccctggctgggtccacggtaggcgtgagcctatctttgtgaagcctcgagatgctttctctgacggcgattcagcccttcagctcggggagctgtctgaatccggctccgtcatcgagtttgaccggacaagaaatgctccggcggtcgacgcccctgttgacttgacggcttcgaacaaggccctctctgtggttgatcctttcgaatttgccgaactcaagcgcccgcgtttttccgcacaagccttaattgaccgaggcggtccacttgccgacgtccatgcaaaaataaagaaccgggtatatgaacagtgcctccaggcttgtgagcctggcagtcgcgcaaccccagccaccaagaagtggctcgataaaatgtgggatagggtggacatgaagacttggcgctgtacctcgcagttccaagctggtcgcattcttgcgtcccttaaattcctccctgacatgattcaggacacaccacctcctgttcctaggaagaatcgggctagtgataatgccggcctgaagcgactggtggcgcagtgggacagaaaattgagtgcaacccccccttcaaaaccggttggaccaacacttgaccaaattgtcccttcgcccacgggtacccagcaagaagatgtcacttcctccgatgggccatctcatgcgccggatcctcctagtcgaatgagcacgagtgggagttggaagggccttgtgctctctggtacccgtctcgcggggtccattagtcagcatttcatgacatgggtttttgaggttttctcccatctcccagcttttgcactcacacttttctcgccgaggggctctatggcttcaggtgattggatgtttgcaggtgttgttttacttgctctcctgctctgtcgttcttacccagtattcgggtgccttcccttattgggtgtcctttctggctctgtgcggcgcgttcgtctgggggtttttggttcttggatggctttcgctgtatttttattctcgactccatccaacccagtcggttcttcttgtgaccacgattcgccggagtgtcatgctgagcttttggctcttgagcagcgccaactttgggaacctgtgcgcggccttgtggtcggcccctcgggcctcttatgtgtcattcttggcaagctactcggtgggtcacgttatctctggcatgttttcttacgtttatgcatgcttgcagatttggccctttctcttgtttatgtggtgtcccaggggcgttgtcacaagtgttggggaaagtgtataagaacagctccggcggaggtggctctcaatgtgttccctttctcgcgcgctacccgtagctctcttgtgtccttgtgcgatcggttccaagcgccaaaaggggttgatcctgtgtacttggcaacgggttggcgcgggtgttggtgtggtgagagtcccattcatcaaccgcaccaaaaacccatagcttatgctaatctggatgaaaagaaaatatctgcccaaacggtggttgctgtcccacacgatcccagtcaggccatcaagtgcctgaaagttttgcaggcgggaggagccatcgtggaccagcccacacctgaggtcgtccgtgtatccgaaatccccttctcagccccatttttcccaaaagttccagtcaacccagattgtaaggttgtggtggattcggacacttttgtggctgcggttcgctgcggctactcgacagcacaactggtcttaggccagggcaactttgccaagttaaatcagattcccctcaggagctctatctccaccaaagcgattggcggggcctcttacacccttgctgtggctcaagtttctgtgtggactcttgttcacttcatcctcggtctttggttcacgtcaccccaagtgtgtggccgaggaacctctgactcatggtgttcaaatcctttttcataccctacctatggccccggggttgtgtgctcctctcgactttgtgtgtctgccgacggggtcactctaccattgttctcagccgtggctcaactctccggtagagaggtggggatttttattttggtactcgtctccttgatggctttggcccaccgcatggcccttaaggcagacatgttgatggtctttctggctttttgtgcttacgcctggcccatgagctcctggttgatttgcttctttcctacactcttgaagtgggttaccctccaccctcttactatgctttgggtgcactcattcttggtgttttgtctgccagcagccggcatcctctcactagggataactggccttctttgggcggttggccgctttactcaggttgccggaattattacaccttatgacatccaccagtacacctctgggccacgtggcgctgcagctgtggccacagccccagaaggcacttatatggccgccgtccggagagctgctttaactgggcgaactttaatcttcaccccgtctgcagttggatcccttctcgaaggagctttcaggactcataaaccttgtcttaacaccgtaaatgttgtaggctcttcccttggttccggcggggttttcactattgacggcagaaaaattgttgtcactgctgcccatgtgttgaacggcgacacagctagagtcaccggcgactcctacaaccgcatgcatactttcaagaccaatggtgattatgcctggtcccatgctgatgactggcggggcgttgcccctgcggtcaaggttgcgaaggggtaccgcggtcgtgcctactggcaaacatcaactggcgtcgaacccggtgttattggggaagggttcgccttctgtttcaccacctgtggcgattcggggtcacccgtcatctcagaatccggtgatctcattggaatccataccggttcaaataaacttggttctggtcttgtgacaacccctgaaggggagacatgtaccatcaaagaaaccaagctctctgacctctccagacatttcgcaggcccaagcgttcctcttggggatattaaattgagtccagccatcatccctgatataacatccattccgagtgacttggcatcgctcctatcctccgtccctgtagtggaaggcggcctctcgaccgttcaacttttgtgtgtctttttcctactttggcgcatgatgggccatgcctggactcccattgttgccgtgggtttctttttactgaatgaaattcttccagcagttttggtccgagccgtgttttcttttgcactttttgtgcttgcatgggccaccccctggtctgcacaggtgttgatgattagactcctcacggcatctctcaaccgcaataagctttctctggcgttctacgcactcgggggggtcgtcggtttggccgctgaaatcgggacttttgctggcaaattgcctgaattgtctcaaaccctttcgacatactgcttcttacctagggtccttgctatgaccagttgtgttcccaccatcatcattggtggactccatgccctcggtgtaattctgtggttgttcaaataccggtgcctccacaacatgctcgttggtgatgggagtttttcaagcgccttcttcctacggtattttgcagagggtaatctcagaaaaggtgtttcgcagtcctgtggcatgaataacgagtccctgacggctgctttggcttgcaagttgtcacgggctgaccttgattttttgtccagcttaacgaacttcaagtgctttgtatctgcttcgaatatgaaaaatgctgccggccagtacattgaagcggcgtatgccaaggccctgcgccaagagttggcgtctctagttcagattgacaaaatgaaaggagttttgtccaaactcgaggcctttgctgaaacagctactccgtcccttgacataggtgacgtgattgttctgcttgggcaacatccacacggatctatccttgatattaatgtggggactgagaggaaaactgtgtccgtgcaagagacccggagcctaggcggttccaaattcagtgtttgtactgtcgtgtctaacacacccgtggacgccttaaccggcatcccactccagacaccaacccctctttttgagaatggtccgcgtcatcgcagcgaagaagacgatcttaaagtcgagaggatgaagaaacactgtgtgcccctcggcttccacaacatcaatggcaaagtttactgcaagatttgggacaagtccaccggtgacaccttttacacggatgattcccggtatacccacgaccatgctcttcaggacaggtcagccgactacagagacagggactatgagggtgtgcaaaccgccccccaacagggatttgatccaaagtctgaaacccctgtcggcactgttgtgatcggcggtattacgtataacaggtatctgactaagggtaaggaggttctggttcccaagcccgacaattgccttgaagctgccaagctgtctcttgagcaagctctcgctgggatgggccaaacttgcgaccttacagctgccgaggtggaaaagttaaagcgcatcatcagtcaactccaaggtttgaccactgaacaggctttaaactgctagccgctagcggcttgacccgctgtggccgcggcggcttagttgttactgaaacggcggtaaaaattgtaagataccacagcagaactttcaccttgggccctttggacctaaaagtcgcttctgaggtggaggtgaagagatcaactgagcagggccacgctgttgtggcaaacctatgttctggtgttgtattgatgagacctcacccaccgtcccttgttgacgttcttctgaaacccggacttgacacaacacccggcattcaaccggggcatggggccgggaatatgggcgtggacggttccatttgggattttgaaaccgcacccacaaaggcagagctcgagttgtccaggcaaataatccaagcatgtgaagtcaggcgcggggatgccccgaacctccaactcccttacaagctctgtccagttaggggggatcctgagcggcataaaggccgccttatcaataccaggtttggagatttgccttacaagactcctcaagacaccaagtccgcaatccacgcggcttgttgcctgcaccccaacggggctcccgtgtctgatggtaaatccacattaggcaccactcttcaacacggtttcgagctttatgtccctactgtgccctatagtgtcatggagtaccttgattcacgccctgacaccccctttatgtgtaccaaacatggtacccccgaggctgctgcagaggaccttcgaaaatatgacttatccacccaagggtttgtcctgcctggggtcctacgtctagtacgcagattcatctttggccatattggtaaggcaccgccactgttcctcccatcaacttatcccgccaagaactctatggcagggatcaatggccagaggttcccaacaaaagacgttcagagcatacctgaaattgatgaaatgtgtgcccgcgctgtcaaagagaattggcaaactgtgacgccttgcaccctcaaaaaacagtattgttccaagcctaaaaccaggaccatccttggtaccaacaacttcattgccttggctcacaggtcggcactcagcggtgtcactcaggcgttcatgaaaaaagcctggaagtccccgatcgccttggggaaaaacaaatttaaggagttgcactgcactgtcgccggtaggtgcctcgaggccgacttggcctcttgtgatcgcagcaccccggccatcgtgaggtggtttgttgccaacctcctgtacgaacttgcgggttgtgaagagtacttgccaagctatgtgctcaattgttgccatgacctggtggcaacgcaaaatggcgccttcacaaaacgtggtggcctgtcatctggagaccccgttaccagtgtgtccaacacagtgtattcactggtgatttatgctcagcacatggtgctgtcggctttgaagatgggtcatgaaatcggcctcaagttcctcgaggaacagcttaagttcgaggaccttctcgaaatccagcctatgttggtatattctgatgaccttgtattgtatgctgaaagacccaccttccccaattaccattggtgggttgagcaccttgacctgatgctgggttttaagacagacccaaaaaagactgtcataactgacaaacccagctttcttggctgtagaatcgaagcggggcgacaactggttcccagtcgcgaccgcattctggctgctcttgcatatcacatgaaggcgcagaacgcctcagagtattatgcgtctgctgccgcgattctgatggactcgtgtgcttgcattgaccacgatcctgagtggtatgaggacctcatctgcggcattgctcgatgcgcccgccaggatggctacagtttcccaggcccgccgttcttcatgtccatgtgggagaagctgaaaagtcacaatgaaggcaagaaattccgccactgtggcatttgtgacgccaaggccgatcatgcgtccgcctgtgggcttgatttatgtctgttccactcgcactttcatcaacactgccctgttactctgagctgcggccatcatgccggttcaaaagaatgcccacagtgtcagtcacctgttggagctggtaaatccccccttgatgcagtgctgaaacaaatcccgtacaaaccccctcgtcctgtcatcatgaaggtggacaataaaacaacgacccttgacccgggaaggtatcagtcccgtcgaggtcttgttgctgtcaagaggggtattgcaggcaatgaggttgatctcgctgatggagactatcaggtggtgccccttttgccaacctgcaaagacataaacatggtgaaggtggctgtcaatgtgctactcagcaagttcatagtggggccgccaggttccggcaagacgacctggctactgagtcaagttcaggatgatgatgtcatctatacacctacccatcagaccatgttcgacatagtcaatgccctcaaagtctgtaggtattccatcccagtggcttcagggctccctttcccaccgcccgccagatctggaccgtgggttaggctcgttgctagcgggcacatccctggccgaatatcataccttgacgaggccggatattgcaatcatctggatattctcagactgctttctaagacacctcttgtgtgtttgggtgaccttcagcaacttcaccctgtcggctttgactcttactgttatgtttttgatcacatgcctcataagcagctgaccactatttatagatttggccccaatatctgttctgccattcaaccttgttacagggaaaaactcgaatctaaggccaggaacactagggtggtttttactgcccggcccgtggcttttggtcaggtgttgacgccatatcataaagaccgcaccggctcagctataaccatagattcatcccagggagccacctttgatgttgtgacgctgcatttgccgtcgccagattccctgaacaaatcccgggctcttgtagctatcactcgggcaaggcatgggttgttcatctatgaccctcataaccaactccggaagtttttcaacttaacacctgagcgcactgattgcaacctcgtgttcaaccgcggggacgagttggtagttttgaatgcagataatgcagtcacgactgtggctaaggttctggaggcgggcccgtctcggtttcgagtatcagatccaaggtgcaagtctcttttagccgcttgctcggccagtctggaaggaggctgcatgccactgccgcaagtggcacataatctggggttttacttctctccagatagtccagcatttgcacctctgccaaaagagctggcaccacattggccggttgttactagtcagaacaaccaggcatggcccgaccgacttgtcgctagtatgcggccagttgatgcccgctacagcaagcctatggtcggtgcagggtatgtggttgggccatccactttccttggcactcctggtgtggtgtcatactatctcacgctgtacatcaggggtgagccccaggccttgccggagacactcgtctcaacgggacgtatagctactgattgtcgagagtatctcgacgcagctgaggaagaagcagctaaagaactccctcacgcattcattggcgatgtcaaaggtactacagtgggggggtgtcaccacattacgtcaaaataccttcccaggtccttgcccaaggactccgttgccgtggtcggagtgagttcgcccggcaaggctgccaaagccgtgtgtactctcaccgatgtgtatcttcccgagctccggccatatttgcaaccggaaacagcatcgaaatgctggaaacttaaactagacttcagggatgtcagactgatggtctggaaaggagccaccgcatatttccaactggaggggctcacatggtcagcgctgcccgactatgccaggttcattcagctaccaaaagatgccgttgtgtacattgatccgtgcataggaccggcaacagccaaccgtaaggttgtgagaactacagattggcgagctgacctggcagtgacaccgtacgactacggtgctcagagcattttgactacagcctggttcgaggaccttgggccgcagtggaagattttagggttgcaacccttcaagcgggcatttggctttgaaaacactgaggattgggcgatccttgcacgtcgcatgaacgacggcaaggactacactgactataactggaattgtgttcgacaacgcccacatgccatctacggacgtgcccgtgaccatacgtatcactttgcccctggcactgaactgcaagtagagctaggtaaacctcggctaccgcctgagcaagtaccgtgaatctagagtgatgcaatggggtcactgtggagtaaaatcagtcaactgttcgtggatgccttcactgagttccttgttagtgtggttgatattgtcatcttccttgccatactgtttgggttcaccgttgcagggtggttactggtcttttttctcagagtggtttgctccgcgcttctccgttcgcgctctgccattcactcttccgaactatcgaaggtcctatgagggcttgctacctaattgcagaccggatgttccacaattcgcattcaagcacccattgggtatgttttggcacatgcgagtttcccacttaattgacgaaatggtctctcgccgcatttatcagaccatggaacattcaggtcaagcggcctggaagcaggtggttagtgaagctactctcacaagactgtcaaagctcgacatagttctccacttccaacacctggccgcaatagaggcggattcttgccgcttcctcagctcacgacttgtgatgctgaaaaatcttgctgttggcaatatgagcctacagtacaacaccacgttggaccgcgttgagctcatcttcccaacaccaggtacgaggcccaaattgaccgattttagacaatggctcgtcagtgtgcacgcttctattttttcctctgtggcctcatcagttaccttgttcatagtgctttggcttcgaattccagctctacgctatgtttttggtttccattggcccacggcaacacatcattcgagctaaccatcaattactctatatgtaagccctgtctcaccagtcaagcggctcgacaaaggctcgaacccggtcgcaacatgtggtgcaaaatagggcacaccacgtgtgaggagcgtgaccatgatgagttgtcaatgaccattccgtccgggtacgataacctcaaacttgagggttattacgcttggctggcttttttgtccttttcttacgcagcccaatttcatccagagttgttcggaatagggaatgtgtcacgcgtcttcgtggataaacggcaccagttcatctgtgccgagcacgacggacaaaattcaaccgtatccaccgaacataatatttccgcattgtatgcggcgtactaccaccaccaggtagacgggggcaattggttccacctggaatggctgcggcctttcttttcctcctggctagtactcaatatttcatggtttctgaggcgttcgcctgcaagccctgtttctcgacgcatttatcagatattgagaccaacacgaccgcggctgccggtttcatggtccttcaggacatcaactgtttccacagtggctcagaggcacaaacgactggtcccatcagaaagtcgtcccaatgccgtgaagccgtcggcactccccagtacatcaagataacggccaatctgaccgacgaatcatacttgtacaacgcggacttgctgatgctttctgcgtgccttttctacgcctcagagatgagcgagaaaggctttaaagtcatttttgggaatgtctctggcgttgtctccgcttgtgtcaattttacagattatgtggcccatctgacccaacatacccagcagcatcatttggtaaccaatcacattcggttgctacatttcctgacaccatcagcgatgaggtgggctacaaccattgcttgtttgttcgccattctcttggcgatatgagatgttctcacaaatcggggtgtttcttgactccgcactcttgcttttggtggttttttttgctgtgtaccggcttgtcctggtcctttgccgatggcaacggcgacagctcgacataccaatatatatataacttgacgatatgcgagctgaatgggaccgaatggctgtctgaccattttaattgggcagtcgagacttttgtgctctacccagtggcgactcatatcctctcactgggtttcctcacgacaagtcatttccttgatgcgttcggtcttggagctgtgtcaattacagggttttgtggcgggcggtacgtgctcagcagcgtgtacggcgcttgtgcactagcagcgctcgtatgttttgttatccgtgctgccaaaaattgtatggcttgccgctatgctcgtacccggtttaccaacttcattgtggatgaccgggggagaatccatcggtggaggtctccaatagtggtggaaaaattaggtaaagctgacgtcggcggcgaccttgtcaccatcaaacatgtcatcctcgaaggagtcaaagctcaacccttgacgaggacttcggccgagcaatgggaggcctagataatttctgcaacgatcccaccgccgcacaaaagcttgtgctagcctttagcatcacgtatacacccatcatgatatacgcccttaaggtgtcacgcggccgactcttggggttgttgcacatcttgatatttctgaactgttctttcacgttcggatacatgacatatatgcattttgaatccaccaaccgtgtcgcgcttaccatggggggcgttgtcgcccttctgtggggcgtttatagtttcatagagtcatggaagtttatcacttccagatgcagattgtgttgcctaggccggcgatacattctggcccctgcccaccacgtagaaagtgctgcaggcctccatccgatcccagcgtctggtaaccaagcatacgctgtgagaaagcccggactaacatcagtgaacggcactctggtaccaggacttcgaggcctcgtgctgggcggcaaacgagctgttaaacgaggagtggttaacctcgtcaagtatggccggtaaaaaccagagccagaagaaaaagaaaaatccagctccaatggggaatggccagtcagtcaatcaactgtgccagctgctgggcacaatgataaagtcccagcgccagcgacccaggggaggacaggctaaaaagaaaaagcctgagaagccacatttccccctggctgctgaagatgacgtccggcaccatctcacccagaccgagcgctccctttgcttgcaatcgatccagacggcttttaatcaaggcgcaggaactgcgtcgctttcatccagcgggaagatcggttttcaggttgagtttatgctaccggttgctcatacagtgcgcctgattcgcgtgacttctacatccgccggtcaggatgcaaattaatttgatagtcaggtgaatggccgcgattggcgtgtggcctctgagtcacctattcaattagggcgatcacatgggggtcagacttaattaggcaggaaccatgtgaccgaaatt

(19) United States
(12) Patent Application Publication
(10) Pub. No.: US 2026/0191945 A1
(43) Pub. Date: Jul. 9, 2026

(54) METHODS AND COMPOSITIONS FOR VACCINATING PIGLETS AGAINST PRRS-1 VIRUS

(71) Applicant: Zoetis Services LLC, Parsippany, NJ (US)

(72) Inventors: Monica BALASCH SANUY, Barcelona (ES); Marta CABANA SUMSI, Barcelona (ES); Jay Gregory CALVERT, Otsego, MI (US); Lucas Park TAYLOR, Greensboro, NC (US)

(21) Appl. No.: 19/134,908

(22) PCT Filed: Dec. 4, 2023
(86) PCT No.: PCT/US2023/082226

§ 371 Date: Jun. 2, 2025

Related U.S. Application Data
(60) Provisional application No. 63/386,218, filed on Dec. 6, 2022.

(51) Int. Cl. A61K 39/12; A61K 39/00; A61P 31/14; A61P 37/04; C12N 7/00

(52) U.S. Cl. CPC A61K 39/12; A61P 31/14; A61P 37/04; C12N 7/00; A61K 2039/543; A61K 2039/545; A61K 2039/552; A61K 2039/6006; C12N 2770/10021; C12N 2770/10034

(57) ABSTRACT

This disclosure provides a vaccine comprising a modified live PRRS-1 virus attenuated in cells expressing porcine CD163 for use in inducing protective immunity in a piglet that is older than 60 hours of age, wherein said vaccine is administered to said piglet intranasally.

### FIELD OF THE INVENTION

This invention is generally in the field of vaccinating piglets against Porcine Reproductive and Respiratory Syndrome (PRRS) virus.

### BACKGROUND OF THE INVENTION

PRRS is characterized by abortions, stillbirths, and other reproductive problems in sows and gilts, as well as respiratory disease in young pigs. The causative agent is the PRRS virus (PRRSV), a member of the family Arteriviridae and the order Nidovirales. The nidoviruses are enveloped viruses having genomes consisting of a single strand of positive polarity RNA. The genomic RNA of a positive-stranded RNA virus fulfills the dual role in both storage and expression of genetic information.

In the late 1980's, two distinct genotypes of the virus emerged nearly simultaneously, one in North America and another in Europe. PRRS virus is now endemic in nearly all swine producing countries and is considered one of the most economically important diseases affecting the global pork industry. European PRRS is generally denominated “type 1” to distinguish it from distantly related North American or “type 2” PRRS (PRRS-2). The various subtypes of European PRRS, referred to as PRRS-1 virus (all of which can be protected against in all aspects of the present invention) are further elaborated in M. P. Murtaugh et al., Virus Research, Vol 154, pp. 18-30, 2010; and M. Shi et al., Virus Research, vol 154 pp. 7-17, 2010.

Vaccines based on modified live PRRS-1 virus have been commercially available. For example, SUVAXYN® PRRS MLV contains modified live PRRS-1 virus grown in cells expressing porcine CD163. This virus is safe and effective for intramuscular administration to piglets as young as one day of age. See, for example, U.S. Pat. No. 11,090,376. It has been demonstrated that SUVAXYN® PRRS MLV (containing PRRS-1 subtype 1 virus) cross-protects against challenges with PRRS-1 subtype 2 and subtype 3 viruses.

The present inventors have discovered that it is possible to induce protective immunity in young piglets by intranasally administering modified live PRRS-1 virus grown in cells expressing porcine CD163 to these piglets.

### SUMMARY OF INVENTION

In one aspect, the invention provides a vaccine comprising a modified live PRRS-1 virus attenuated in cells expressing porcine CD163 for use in inducing protective immunity in a piglet that is older than 60 hours of age, wherein said vaccine is administered to said piglet intranasally.

In certain embodiments, the genome of said modified live PRRS-1 virus comprises an RNA molecule that is SEQ ID NO: 1 or that is at least 75% identical to SEQ ID NO: 1, and wherein further,
a) the amino acid sequence encoded by ORF1a of the genome of the modified live PRRS-1 virus in said vaccine contains: S, A, or T, preferably S, at amino acid position 19; Y F, or W, preferably Y, at amino acid position 157; D or E, preferably D, at amino acid position 268; H, R, or K, preferably H, at amino acid position 294; Y, F, or W, preferably Y, at amino acid position 416; S, A, or T, preferably S, at amino acid position 742; L, I, M, or V, preferably L, at amino acid position 884; P at amino acid position 908; K, R, or H, preferably K, at amino acid position 916; K, R, or H, preferably K, at amino acid position 977; S, A, or T, preferably S, at amino acid position 1138; F, Y, or W, preferably F, at amino acid position 1160; S, A, or T, preferably S, at amino acid position 1500; R, K, or H, preferably R, at amino acid position 2094; P at amino acid position 2254; and L, I, M, or V, preferably L, at amino acid position 2290; and
b) the amino acid sequence encoded by ORF1b of the genome of the modified live PRRS-1 virus in said vaccine contains: S, A, or T, preferably S, at amino acid position 567; and H, R, or K, preferably H, at amino acid position 912; and
c) the amino acid sequence encoded by ORF2a of the genome of the modified live PRRS-1 virus in said vaccine contains: L, I, M, or V, preferably L, at amino acid position 22; F, Y, W, preferably F, at amino acid position 88; M, L, I, or V, preferably M, at amino acid position 94; and F, Y, or W, preferably F, at amino acid position 95; and
d) the amino acid sequence encoded by ORF2b of the genome of the modified live PRRS-1 virus in said vaccine contains: L, I, M, or V, preferably L, at amino acid position 47; and
e) the amino acid sequence encoded by ORF4 of the genome of the modified live PRRS-1 virus in said vaccine contains: T, S, or A, preferably T, at amino acid position 151 and
f) the amino acid sequence encoded by ORF5 of the genome of the modified live PRRS-1 virus in said vaccine contains: F, Y, or W, preferably F, at amino acid position 20; and D or E, preferably D, at amino acid position 37; and
g) the amino acid sequence encoded by ORF5a of the genome of the modified live PRRS-1 virus in said vaccine contains: V, L, I, or M, preferably V, at amino acid position 18; and R, H, or K, preferably R, at amino acid position 35.

In a subset of embodiments, the in the vaccine used as disclosed herein: the amino acid sequence encoded by ORF1a of the genome of the modified live PRRS-1 virus in said vaccine further contains Y at amino acid position 24; and A at amino acid position 156; and the amino acid sequence encoded by ORF3 of the genome of the modified live PRRS-1 virus in said vaccine further contains S at amino acid position 52.

In certain embodiments, applicable to any of the vaccines disclosed above, the genome of said modified live PRRS-1 virus comprises an RNA molecule that is at least 90% identical to SEQ ID NO: 1, preferably, at least 95% identical to SEQ ID NO: 1. In most preferred embodiments, the genome of said modified live PRRS-1 virus is identical to SEQ ID NO: 1 or at least 50% (or at least 60%, or at least 70% or at least 80% or at least 90% or at least 95% or 100%) of the nucleotides differing from the corresponding nucleotides in SEQ ID NO: 1 result in silent mutations or conservative substitutions or any combination thereof.

In any of the embodiments of the vaccine comprising the modified live PRRS-1 virus, the vaccine is administered intranasally to piglets that are older than 60 hours of age, including at least 6 4 hours of age, or at least 68 hours of age, or at least 72 hours of age, or at least 76 hours of age, or at least 80 hours of age, or at least 84 hours of age. At the same time, in certain embodiments, the piglet is about 21 days of age or younger, or about 14 days of age or younger, or about 10 days of age or younger, or about 7 days of age or younger, or about 5 days of age or younger.

In certain embodiments, the protective immunity induced by the intranasal administration of the vaccine disclosed herein to the piglets that is older than 60 days comprises at least one of reduced viremia, reduced viral shedding, lung lesion scoring, and lung lesion frequency.

In certain embodiments, the piglet is MDA-positive. In other embodiments, the piglet is MDA-negative. In the embodiments where the piglet is MDA negative said protective response further comprises weight gain compared to unvaccinated infected piglets.

In certain embodiments, said vaccine is a single-dose vaccine.

### DETAILED DESCRIPTION

In order to better explain the invention, the following definitions are provided

The term “about” as applied to a reference number refers to the reference number plus or minus 10 percent of said value, except when the value is provided as a base-exponent expression, (e.g., 10³), then the term “about” refers to the range of the exponent within 10% of the value of the exponent. Using the example of 10³, “about 10³” is between 10^{2.7} and 10^{3.3}.

The terms “singe-dose vaccine” or “vaccine effective as a single dose” or the like refer to the ability of the vaccine to induce protective immunity upon single administration. The duration of such protective immunity is sufficient that the pig is not revaccinated for about six months or at any time prior to the slaughter of said pig, whichever comes first.

The terms “protective immunity” or “protective immune response” or the like refer to the ability of the vaccine to cause a reduction in magnitude, frequency or duration of at least one clinical sign of PRRS-1 virus infection. Such clinical signs include, without limitations, lung lesion magnitude, lung lesion frequency, oral shedding, nasal shedding, and viremia.

The terms “effective amount” or “effective dose” refer to an amount of an antigen that induces a protective immune response in a subject receiving the antigen after then subject is challenged or infected with a virulent PRRS-1 virus. In certain embodiments, the virulent PRRS-1 virus may be PRRS-1 virus subtype 1, or subtype 2, or subtype 3. In more preferred embodiments, the virulent PRRS-1 virus is PRRS-1 virus subtype 1.

The term “vaccine” refers to a composition that contains an effective amount of the antigen.

In one aspect, the invention provides a vaccine comprising a modified live PRRS-1 virus attenuated in cells expressing porcine CD163 for use in inducing protective immunity in a piglet that is at least three days old, wherein said vaccine is administered to said piglet intranasally.

The methods of creating cells expressing porcine CD163 cells are known in the art. Suitable starting points include without limitations monkey kidney cell line MA-104 cells, baby hamster kidney BHK21 cells, and porcine kidney cells PK0809 cells. These cells can be transfected with a nucleic acid sequence encoding porcine CD163. The resulting clones, MARC-145 cells, BHK21-C12-26, and PK-9 cells are suitable non-limiting examples of cells expressing porcine CD163. These cells have been disclosed, for example, in U.S. Pat. No. 11,090,376. Also see U.S.

U.S. Pat. No. 9,102,912 referring to assignment of the mammalian CD163 surface protein as the normal PRRS virus cell receptor).

As an illustration, to demonstrate that a particular genetically modified strain is attenuated an experiment described as follows may be used. This illustration applies both to PRRS-1 and PRRS-2.

At least 10 gilts per group are included in each trial, which are derived from a PRRSV-free farm. Animals are tested free of PRRS virus specific serum antibodies and negative for PRRSV. All animals included in the trial are of the same source and breed. The allocation of the groups to animals is randomized.

Challenge is performed at day 90 of pregnancy with intranasal application of 1 ml PRRSV with 10⁵ TCID₅₀ per nostril. There are at least three groups for each test setup: One group for wild type virus; one test group for challenge with the possibly attenuated virus; and one strict control group.

The study is deemed valid when the strict controls stay PRRSV-negative over the time course of the study and at least 25% less live healthy piglets are born in the wild type challenged group compared to the strict controls.

Attenuation, in other words less virulence, is defined as the statistically significant change of one or more variables determining reproductive performance or other symptomology:

Significant reduction in at least one of the following variables for the test group (possibly attenuated virus) compared to the unmodified parental strain infected group would be an indication of attenuation:
a) frequency of stillborns
b) abortion at or before day 112 of pregnancy
c) number of mummified piglets
d) number of less lively and weak piglets
e) pre-weaning mortality

Furthermore a significant increase in one of the following variables for the test group compared the unmodified parental strain infected group is preferred:
f) number of piglets weaned per sow;
g) number of live healthy piglets born per sow.

In the alternative, respiratory symptoms and other symptoms of PRRSV infection could be examined to establish attenuation.

In certain embodiments, the modified live PRRS-1 virus is the same virus that is used in SUVAXYN® PRRS MLV. This virus is the progeny of PRRS-1 virus strain 96V198 attenuated in cells expressing porcine CD163. The genome of the virus used in SUVAXYN® PRRS MLV comprises SEQ ID NO: 1.

In identifying the preferred encoded amino acids, the corresponding/original amino acids of the wild isolate of strain 96V198 are also shown by parenthesis, thus passage zero (wild type) is compared to passage 49 (the genome of PRRS-1 virus in SUVAXYN® PRRS MLV, SEQ ID NO:1). Thus, the amino acid sequence encoded by ORF1a of SEQ ID NO 1 contains:
S at amino acid position 19 (N);
Y at amino acid position 24 (F);
A at amino acid position 156 (T);
Y at amino acid position 157 (IH);
D at amino acid position 268 (N);
H at amino acid position 294 (Y);
Y at amino acid position 416 (C);
S at amino acid position 742 (P);
L at amino acid position 884 (F);
P at amino acid position 908 (S);
K at amino acid position 916 (E);
K at amino acid position 977 (E);
S at amino acid position 1.138 (P);
F at amino acid position 1160 (L);
S at amino acid position 1500 (P);
R at amino acid position 2094 (Q);
P at amino acid position 2254 (S); and
L at amino acid position 2290 (F).

The amino acid sequence encoded by ORF1b of SEQ ID NO: 1 contains:
S at amino acid position 567 (N); and
H at amino acid position 912 (Q).

The amino acid sequence encoded by ORF2a of SEQ ID NO: 1 contains:
L at amino acid position 22 (S);
F at amino acid position 88 (V);
M at amino acid position 94 (1); and
F at amino acid position 95 (L).

The amino acid sequence encoded by ORF2b of SEQ ID NO: 1 contains:
L at amino acid position 47 (F).

The amino acid sequence encoded by ORF3 of SEQ ID NO: 1 contains:
S at amino acid position 52 (T).

The amino acid sequence encoded by ORF4 of SEQ 0 NO: 1 contains:
T at amino acid position 151 (I).

The amino acid sequence encoded by ORF5 of SEQ ID NO: 1 contains:
F at amino acid position 20 (L); and
D at amino acid position 37 (N).

The amino acid sequence encoded by ORF5a of SEQ ID NO: 1 contains:
V at amino acid position 18 (A); and
R at amino acid position 35 (Q).

Nucleic acid sequence identities according to any of the embodiments described herein can be evaluated using any of the variety of sequence comparison algorithms and programs known in the art. For sequence comparison, typically one sequence acts as a reference sequence (e.g., a sequence disclosed herein, such as SEQ ID NO: 1), to which test sequences are compared. A sequence comparison algorithm then calculates the percent sequence identities for the test sequences relative to the reference sequence, based on the program parameters.

The percent identity of two nucleic acid sequences can be determined for example by comparing sequence information using the computer program GAP, i.e., Genetics Computer Group (GCG; Madison, WI) Wisconsin package version 10.0 program, GAP (Devereux et al. (1984), Nucleic Acids Res. 12: 387-95). In calculating percent identity, the sequences being compared are typically aligned in a way that gives the largest match between the sequences. The preferred default parameters for the GAP program include: (1) The GCG implementation of a unary comparison matrix (containing a value of 1 for identities and 0 for non-identities) for nucleotides, and the weighted amino acid comparison matrix of Gribskov and Burgess, ((1986) Nucleic Acids Res. 14: 6745) as described in Atlas of Polypeptide Sequence and Structure, Schwartz and Dayhoff, eds., National Biomedical Research Foundation, pp. 353-358 (1979) or other comparable comparison matrices; (2) a penalty of 8 for each gap and an additional penalty of 2 for each symbol in each gap for amino acid sequences, or a penalty of 50 for each gap and an additional penalty of 3 for each symbol in each gap for nucleotide sequences; (3) no penalty for end gaps; and (4) no maximum penalty for long gaps.

Sequence identity and/or similarity can also be determined by using the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (BESTFIT, FASTA, and TFASTA in the Wisconsin Genetics Software Package, Genetics Computer Group, 575 Science Drive, Madison, Wis.).

Another example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xᵤ set to 16, and X_{g} set to 40 for database search stage and to 67 for the output stage of the algorithms. Gapped alignments are triggered by a score corresponding to about 22 bits.

Since the genetic code is degenerate, a homologous nucleotide sequence can include any number of “silent” base changes, i.e. nucleotide substitutions that nonetheless encode the same amino acid.

The skilled person will further acknowledge that alterations of the nucleic acid sequence resulting in modifications of the amino acid sequence of the protein it codes may have little, if any, effect on the resulting three-dimensional structure of the protein. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in the substitution of one negatively charged residue for another, such as aspartic acid for glutamic acid, or one positively charged residue for another, such as lysine for arginine, can also be expected to produce a protein with substantially the same functional activity.

The following six groups each contain amino acids that are typical conservative substitutions for one another: [1] Alanine (A), Serine (S), Threonine (T); [2] Aspartic acid (D), Glutamic acid (E); [3] Asparagine (N), Glutamine (Q); [4] Arginine (R), Lysine (K), Histidine (H); [5] Isoleucine (1), Leucine (L), Methionine (M), Valine (V); and [6] Phenylalanine (F), Tyrosine (Y), Tryptophan (W), (see, e.g., US Patent Publication 20100291549).

In certain embodiments, the genome of the modified live virus comprises SEQ ID NO: 1 or is at least 75% identical (or at least 80% identical, or at least 85% identical, or at least 90% identical, or at least 95% identical, or at least 98% identical, or 100% identical) to SEQ ID NO: 1.

In further embodiments, the genome of the modified live virus comprises SEQ ID NO: 1 or is at least 75% identical (or at least 80% identical, or at least 85% identical, or at least 90% identical, or at least 95% identical, or at least 98% identical, or 100% identical) to SEQ ID NO: 1, wherein further,
a) the amino acid sequence encoded by ORF1a of the genome of the modified live PRRS-1 virus in said vaccine contains:
   S, A, or T, preferably S, at amino acid position 19;
   Y, F, or W, preferably Y, at amino acid position 157;
   D or E, preferably D, at amino acid position 268;
   H, R, or K, preferably H, at amino acid position 294;
   Y, F, or W, preferably Y, at amino acid position 416;
   S, A, or T, preferably S, at amino acid position 742;
   L, I, M, or V, preferably L, at amino acid position 884;
   P at amino acid position 908;
   K, R, or H, preferably K, at amino acid position 916;
   K, R, or H, preferably K, at amino acid position 977;
   S, A, or T, preferably S, at amino acid position 1138;
   F, Y, or W, preferably F, at amino acid position 1160;
   S, A, or T, preferably S, at amino acid position 1500;
   R, K, or H, preferably R, at amino acid position 2094;
   P at amino acid position 2254; and
   L, I, M, or V, preferably L, at amino acid position 2290; and
b) the amino acid sequence encoded by ORF1b of the genome of the modified live PRRS-1 virus in said vaccine contains:
   S, A, or T, preferably S, at amino acid position 567; and
   H, R, or K, preferably H, at amino acid position 912; and
c) the amino acid sequence encoded by ORF2a of the genome of the modified live PRRS-1 virus in said vaccine contains:
   L, I, M, or V, preferably L, at amino acid position 22;
   F, Y, W, preferably F, at amino acid position 88;
   M, L, I, or V, preferably M, at amino acid position 94; and
   F, Y, or W, preferably F, at amino acid position 95; and
d) the amino acid sequence encoded by ORF2b of the genome of the modified live PRRS-1 virus in said vaccine contains:
   L, I, M, or V, preferably L, at amino acid position 47; and
e) the amino acid sequence encoded by ORF4 of the genome of the modified live PRRS-1 virus in said vaccine contains:
   T, S, or A, preferably T, at amino acid position 151 and
f) the amino acid sequence encoded by ORF5 of the genome of the modified live PRRS-1 virus in said vaccine contains:
   F, Y, or W, preferably F, at amino acid position 20; and
   D or E, preferably D, at amino acid position 37; and
g) the amino acid sequence encoded by ORF5a of the genome of the modified live PRRS-1 virus in said vaccine contains:
   V, L, I, or M, preferably V, at amino acid position 18; and
   R, H, or K, preferably R, at amino acid position 35.

These amino acids are non-conservative substitutions compared to the wild-type parent PRRS-1 virus.

In additional embodiments, ORF1a protein encoded by the genome of the modified live PRRS-1 virus in the vaccine contains the following amino acids, in addition to the amino acids specified above:
a) in ORF1a: Y at amino acid position 24; and A at amino acid position 156; and
b) in ORF3: S at amino acid position 52.

In a more particular subset of embodiments the genome of the modified live virus comprises SEQ ID NO: 1 or is at least 75% identical (or at least 80% identical, or at least 85% identical, or at least 90% identical, or at least 95% identical, or at least 98% identical, 99% identical) to SEQ ID NO: 1, wherein,
b) the amino acid sequence encoded by ORF1a of the genome of the modified live PRRS-1 virus in said vaccine contains:
   S at amino acid position 19;
   Y at amino acid position 24;
   A at amino acid position 156
   Y at amino acid position 157;
   D at amino acid position 268;
   H at amino acid position 294;
   Y at amino acid position 416;
   S at amino acid position 742;
   L at amino acid position 884;
   P at amino acid position 908;
   K at amino acid position 916;
   K at amino acid position 977;
   S at amino acid position 1138;
   F at amino acid position 1160;
   S at amino acid position 1500;
   R at amino acid position 2094;
   P at amino acid position 2254; and
   L at amino acid position 2290; and
b) the amino acid sequence encoded by ORF1b of the genome of the modified live PRRS-1 virus in said vaccine contains:
   S at amino acid position 567; and
   H at amino acid position 912; and
c) the amino acid sequence encoded by ORF2a of the genome of the modified live PRRS-1 virus in said vaccine contains:
   L at amino acid position 22;
   F at amino acid position 88;
   M at amino acid position 94; and
   F at amino acid position 95; and
d) the amino acid sequence encoded by ORF2b of the genome of the modified live PRRS-1 virus in said vaccine contains:
   L at amino acid position 47; and
e) the amino acid sequence encoded by ORF3 of the genome of the modified live PRRS-1 virus in said vaccine contains:
   S at amino acid position 52; and
f) the amino acid sequence encoded by ORF4 of the genome of the modified live PRRS-1 virus in said vaccine contains:
   T at amino acid position 151 and
g) the amino acid sequence encoded by ORF5 of the genome of the modified live PRRS-1 virus in said vaccine contains:
   F at amino acid position 20; and
   D at amino acid position 37; and
h) the amino acid sequence encoded by ORF5a of the genome of the modified live PRRS-1 virus in said vaccine contains:
   V at amino acid position 18; and
   R at amino acid position 35.

Preferably, the at least 50% (or at least 60%, or at least 70%, or at least 80%, or at least 90%, or at least 95%, or at least 98% or 100%) of the nucleotides that differ between the RNA genome of the modified live PRRS-1 virus described herein SEQ ID NO: 1 (outside of the nucleotides encoding the specified amino acids disclosed above) result in
a) silent mutations or
b) conservative substitutions or
c) a combination thereof.

The effective dose of the modified live PRRS-1 virus of the present invention can be determined using known techniques, taking into account factors that can be determined by one of ordinary skill in the art such as the weight of the animal to be vaccinated. The dose amount of virus of the present invention in a vaccine of the present invention preferably ranges from about 10¹ to about 109 TCID₅₀ (Tissue Culture Infectious Dose 50%), more preferably from about 10^{1.5} to about 108 TCID₅₀, more preferably from about 102 to about 10⁶ TCID₅₀, more preferably about 10^{2.2} to about 10^{5.2} TCID₅₀, or from about 10^{2.2} to about 10^{4.2} TCID₅₀ per dose, from about 10^{2.2} to about 10^{5.2} TCID₅₀ per dose.

A suitable dose volume of the vaccines disclosed herein ranges from about 0.25 ml to about 10 ml, and more preferably from about 0.5 ml to about 5 ml or from about 1 ml to about 3 ml or about 2 ml.

Thus, in a particularly preferred set of embodiments, the volume is about 2 ml and the dose is about 10^{2.2} to 10^{5.2} TCID₅₀, e.g., about 10^{2.2} TCID₅₀.

Vaccines of the present invention can be formulated following accepted convention to include acceptable carriers for pigs, such as standard buffers, stabilizers, diluents, preservatives, mucoadhesives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Mucoadhesive polymers have numerous hydrophilic groups, such as hydroxyl, carboxyl, amide, and sulfate. These groups attach to mucus or the cell membrane by various interactions such as hydrogen bonding and hydrophobic or electrostatic interactions. Lectins and thiolated polymers are non-limiting examples of mucoadhesives. Other suitable vaccine vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

Previously, it has been shown (see U.S. Pat. No. 11,090,376) that vaccines containing the modified live PRRS-1 virus are safe when administered to one-day-old piglets. Efficiency of the vaccine has also been demonstrated in one-day-old piglets when the vaccine was administered intramuscularly. However, the vaccine was not effective when it was administered intranasally.

The inventors have discovered that when the vaccine is administered intranasally to about three-day-old piglets, the vaccines were effective. Accordingly, the vaccine according to any of the embodiments disclosed above, may be intranasally administered to a piglet that is older than 60 hours of age, including without limitations at least 64 hours of age, or at least 68 hours of age, or at least 72 hours of age, or at least 76 hours of age, or at least 80 hours of age, or at least 84 hours of age. At the same time, in certain embodiments, the piglet that is older than any of the lower age thresholds specified in this paragraph may be younger than about 21 days of age or younger than about 14 days of age, or younger than about 10 days of age or younger than about 7 days of age or younger than about 5 days of age.

The vaccine disclosed herein used according to any of the embodiments of the invention elicits protective immunity when administered intranasally to piglets that are generally older than 60 hours of age as described in the previous paragraph). There are multiple ways to measure protective immunity. Suitable endpoints for measuring protective immunity include viremia, viral shedding, and lung lesions.

As noted above, U.S. Pat. No. 11,090,376 disclosed that intranasal administration of a modified live PRRS-1 virus attenuated in cells expressing porcine CD163 to one day old piglets was ineffective. The inventors of the '376 patent suggested that the lack of efficacy was due to Maternally Derived Antibodies, or MDA. However, if the vaccine is administered to pigs that are at about 3 days of age or older, MDA-status of the piglet does not significantly interfere with the protective immunity induced by the vaccine.

In certain embodiments, induction of protective immunity is measured by at least one of: decreased viremia, decreased viral shedding (may be nasal or oral or oronasal), decreased lung lesion extent, decreased lung lesion frequency). As disclosed in the Examples, both PRRS-1 MDA-positive and PRRS-1 MDA-negative three-day old piglets exhibited reduced viremia, reduced shedding (nasal in PRRS-1 MDA-negative and both nasal and oral in PRRS-1 MDA-positive piglets), lung lesion magnitude and lung lesion frequency after challenge. In addition, PRRS-1 MDA-negative piglets exhibit higher body weight and higher weight gain compared to non-vaccinated piglets challenged with PRRS-1.

Uses of the vaccine as described herein results in onset of immunity at about 21 days after vaccination.

The invention will now be described in the following non-limiting examples.

### EXAMPLES

### Example 1. Evaluation of the Onset of Immunity of SUVAXYN® PRRS MLV in Piglets Vaccinated at Three Days of Age by Intranasal Route

The objective of this study was to determine whether intranasal administration of SUVAXIN® PRRS MLV can elicit protective response if administered intranasally to piglets.

The animals were allocated to treatments and host sows according to the plan provided by a Biometrics Representative (BMR) designated by Zoetis VMRD. Before farrowing, four sows were randomly assigned to two rooms. Treatments were randomly assigned to rooms using a completely randomized design.

Piglets were cross fostered onto host sows following the allocation plan provided by the BMR such that each sow fostered piglets from all sows. Before challenge, sows were removed, and piglets were moved into one room and comingled into a single pen. The experimental unit was the room for the vaccination phase and the animal for the challenge phase. The birth sow was the block using a generalized randomized block design in the challenge phase.

Sows were fed once a day with a commercially available feed suitable for their age and physiological status (lactation). Feed was provided individually once a day. Piglets were suckling from birth until weaning (3 weeks of age). Afterwards, an age-appropriate diet was provided ad libitum. Water was provided ad libitum for both sows and piglets.

Sows were moved to the research facility approximately 10 days before farrowing where they were placed in individual farrowing crates (two rooms) according to the allocation plan.

Piglets were housed with the sows until weaning (just before Challenge), when sows were removed, and piglets from the T01 and T02 were comingled in a unique pen in the same room. On the day of the vaccination (day 0) piglets were cross-fostered, weighed, examined, and determined if suitable for the study. At the time of the challenge, the piglets were 3 or 4 days (fewer than 20% of the piglets) of age.

On Day 0, piglets were vaccinated with saline (Group T01) or the vaccine (Group T02) as described in section 4. Each animal in T02 was administered a 2 mL volume of SUVAAXYN® PRRS MLV (10^{2.2} TCID₅₀ per 2 ml) and each animal in T01 was administered a 2 mL volume of the control material inoculated intranasally with a syringe without a needle (IN) 1 ml in each nostril.

On day 21, the piglets were challenged with virulent PRRS virus strain Olot/91. On the day of challenge, the virus stock (10^{5.8} TCID50/mL) was thawed and diluted 1:9 in cell culture medium to match the target titer of 10^{5.0} TCID50/2 mL. The virus was kept on ice during the challenge process. Pre- and post-challenge titrations were performed in porcine alveolar macrophages (PAM). The challenge material was administered intranasally (IN), 1 mL to each nostril

Frequency distributions of clinical signs prior to challenge and clinical signs following challenge were calculated, separately, for each treatment and time point data was collected. Frequency distributions of whether an animal ever had clinical signs, for each phase (Days DC (reference to pre challenge) and Days>DC (post challenge)), was calculated for each treatment. If an animal ever had a clinical sign (post challenge) was analyzed with a generalized linear mixed model, with a logit link with fixed effect treatment and random effect block. This model did not converge. The Fisher's Exact test was applied.

Viremia: Prior to statistical analysis the RT-qPCR data was transformed using an appropriate logarithm transformation. The transformed data was analyzed using a general linear repeated measured mixed model with fixed effects: treatment, time point, and treatment and time point interaction and random effects: block, and animal within block by treatment (which is the animal term). Pairwise treatment comparisons were made at each time point given that the treatment by time point interaction effect is significant (P≤0.05). Treatment least squares mean and 95% confidence intervals were back transformed for presentation. The area under the curve (AUC) of the transformed data was also calculated for each animal, log transformed and analyzed using a general linear mixed model with fixed effects: treatment and random effects: block. Pairwise treatment comparisons were made given that the treatment was significant (P≤0.05). Treatment least squares mean, standard error, range, and 95% confidence interval were presented. Percentage of days viremic/shedding was also calculated. It was also determined if an animal was ever viremic or ever shed for Days DC (reference to pre-challenge) and Days>DC (post-challenge).

Body weights were analyzed using a general linear repeated measure mixed model with fixed effects: treatment, time point, and treatment and time point interaction and random effects: block, and animal within block by treatment (which is the animal term). Treatment least squares means, 95% confidence intervals, the minimum and maximum was calculated for each time point data are collected. Pair wise treatment comparisons were made between treatment groups given that the treatment effect or treatment by time point interaction was significant. Average daily gain estimates were estimated using parameter-based model estimates. Comparisons of average daily gain by treatment were made.

Percentage of total lung with lesions was calculated using the following formula:

$$Percentage of total lung with lesions = ( 0.1 \times left cranial ) + ( 0.1 \times left middle ) + ( 0.25 \times left caudal ) + ( 0.1 \times right cranial ) + ( 0.1 \times right middle ) + ( 0.25 \times right caudal ) + ( 0.1 \times accessory ) .$$

The arcsine square root transformation was applied to the percentage of total lung with lesions prior to analysis. The transformed lung lesions were analyzed with a general linear mixed model with fixed effect: treatment and random effect: block. Pairwise comparisons were made between treatment groups given that the treatment effect is significant. Back transformed least squares means of percentage of total lung with lesions, their standard errors, and their 95% confidence intervals was calculated as well as the minimums and maximums.

Frequency distributions of lung lesion assessment scores were calculated for each treatment. The scores, normal or not normal, were analyzed using a generalized linear mixed model for binomial data with fixed effect: treatment and random effect: block. As the treatment main effect was significant, then pairwise treatment comparisons were made.

Rectal temperatures were analyzed using a general linear repeated measures mixed model analysis with fixed effects: treatment, time point, and treatment and time point interaction and random effects: block, and animal within block by treatment (which is the animal term). Treatment least squares means, 95% confidence intervals, the minimum and maximum were calculated for each time point. Descriptive statistics, means, standard deviations, and ranges, were calculated for each treatment and day of study, pre-challenge. Frequency distributions of animals with a fever (rectal temperature≥40.5° C.) were calculated for each treatment and time point data is collected. It was determined if an animal ever had fever for Days=DC and for Days>DC. Frequency tables for if an animal ever had fever were calculated for each period, prior to challenge and Days>DC.

Prior to statistical analysis the serology was transformed using an appropriate logarithm transformation. The transformed serology data was analysed using a general linear repeated measured mixed model with fixed effects: treatment, time point, and treatment and time point interaction and random effects: block, and animal within block by treatment (which is the animal term). Pairwise treatment comparisons were made at each time point given that the treatment or treatment by time point interaction effect was significant (P≤0.05). Treatment least square means and 95% confidence intervals were back transformed for presentation. Also, it was determined for each animal whether or not it seroconverted (≥0.4 S/P ratio) at any time during the study. Frequency distributions of whether or not an animal seroconverted were calculated for each treatment at each time point. Descriptive statistics, means, standard deviations, and ranges, were calculated for each treatment and time point pre-challenge.

### Results

### Viremia

The presence of PRRS virus in serum was monitored by means of a RT-qPCR assay performed following local procedures. All pigs were found RT-qPCR PRRSV negative in serum before vaccination (Day 0). On Day 21, before challenge, PRRSV RNA was detected in 100% of the pigs in T02 group. By that time, all T01 pigs remained PRRSV negative. By Day 23, two days post-challenge, 100% of piglets from T01 control group developed viremia and continued viremic during the monitored period after challenge. 100% of animals in T02 maintained viro-positive during the monitored period. Before challenge, on Day 21, the viral load detected in serum was significantly (P<0.0001) higher in the T02 group compared to T01 due to the presence of remaining vaccine virus. After challenge, the viral load detected in serum was significantly (P≤0.0330) lower in the T02 group compared to T01 group in all monitored time-points after challenge corresponding to 2-, 4-, 7- and 10-days post-challenge.

The mean AUC of log viremia up to 10 days post-challenge (from Day 21 to Day 31), as well as confidence interval (Cl) variation and ranges per each treatment are summarized in Table 2. PRRSV viremia as the mean AUC value for the complete period was significantly (p<0.0001) lower in T02 group compared to T01 group with mean values of 7.47±0.14 and of 8.62±0.14 respectively.

TABLE 1
Back-transformed Least Squares Mean PRRSV RNA copies/ml in Serum

| | D21 (before | | | | |
|---|---|---|---|---|---|
| Group | challenge) | D23 | D25 | D28 | D31 |
| T01 | 47.5 | 34586961 | 42978817 | 17209023 | 6720302 |
| T02 | 5432642 | 2139202 | 523266.1 | 344000.4 | 1924833 |
| P value | P < 0.0001 | P < 0.0001 | P < 0.0001 | P < 0.0001 | P = 0.0330 |

TABLE 2
Analysis of AUC PRRSV RNA copies/ml in Serum
Post-Challenge (Days >21)

| Group | LSM | SE | Lower 95% CI | Upper 95% Cl | Range |
|---|---|---|---|---|---|
| T01 | 8.62 | 0.14 | 8.34 | 8.91 | 7.51 to 10.08 |
| T02 | 7.47 | 0.14 | 7.19 | 7.76 | 6.05 to 8.59 |

| | |
|---|---|
| P value | P < 0.0001 |

### Lung Lesions

The percentage of lung with lesions for each treatment group is shown in Table 3. Comparison between treatment groups showed significant differences in the % of total lung with lesions (p=0.0023). The percentage of total lung with lesions was lower in IVP vaccinated (T02) group compared to control (T01) group.

TABLE 3
Analysis of Percentage (%) of total lung with lesions

| | | Back transform | | Lower | Upper | |
|---|---|---|---|---|---|---|
| Group | N | LSM | SE | 95% CI | 95% CI | Range |
| T01 | 21 | 2.6 | 0.53 | 1.6 | 3.8 | 0.0 to 9.0 |
| T02 | 22 | 0.7 | 0.27 | 0.2 | 1.3 | 0.0 to 4.8 |

Lung visual score by treatment is summarized in Table 4. At necropsy, 85.7% (18 out of 21) of piglets from T01 control group, had a positive lung visual score (score>0), indicating that PRRSV challenge was successful in inducing lung lesions. The percentage of animals scoring positive (score>0) in the IVP vaccinated (T02) group was of 50.0% (11 out of 22) and significantly lower (P=0.0229) than the control (T01) group. Moreover, none of the animals vaccinated with the IVP (T02) had a lung assessment score of 2 whereas 4.8% (one out of 21) animals from T01 did have a lung assessment score of 2.

TABLE 4
Lung Visual Score by treatment group

| | Visual Score | | | | | | |
|---|---|---|---|---|---|---|---|
| | 0 | | 1 | | 2 | | Total |
| Group | N | % | N | % | N | % | observations |
| T01 | 3 | 14.3 | 17 | 81 | 1 | 4.8 | 21 |
| T02 | 11 | 50 | 11 | 50 | 0 | 0 | 22 |

### Nasal and Oral Shedding

The presence of PRRS virus in Nasal Swab samples was monitored by means of a RTqPCR assay performed following local procedures. All pigs were found RT-qPCR PRRSV negative in nasal swab samples before vaccination (Day<0). On Day 21, before challenge, PRRSV RNA was detected in Nasal Swab samples in 40.9% (nine out of 22) of the pigs in group T02. By that time, all T01 pigs remained PRRSV negative. On Day 23, two days post-challenge, 95.5% of piglets from T01 shed virus by nasal route reaching a peak four days post-challenge (Day 25) with 100% of the piglets from T01 shedding. Even 100% of piglets vaccinated with the IVP (T02) at one or more time points post-challenge (Days>21, see Table 8 percentages of animals with nasal shedding never reached 100% at one time-point (See Table 5). Frequencies of nasal shedding animals, per treatment group at each time-point are detailed in Table 5 and Frequencies of ever shedding are summarized in Table 6.

TABLE 5
Percent of animals with nasal shedding
for each treatment at each day

| Group | D 0 | D 21 (before challenge) | D 23 | D 25 | D 28 | D 31 |
|---|---|---|---|---|---|---|
| T01 | 0 | 0 | 95.5 | 100 | 100 | 85.7 |
| T02 | 0 | 40.9 | 81.8 | 59.1 | 40.9 | 77.3 |

TABLE 6
Frequency Distribution of Ever Shedding in Nasal Swab
Post-Challenge (Days >21)

| | Visual Score | | | | |
|---|---|---|---|---|---|
| | NO | | YES | | |
| Group | N | % | N | % | Total observations |
| T01 | 3 | 14.3 | 17 | 81 | 21 |
| T02 | 11 | 50 | 11 | 50 | 22 |

Before challenge, on Day 21, the amount of viral load detected in Nasal Swab samples was significantly (P=0.0175) higher in the pigs in group T02 compared to the ones in group T01 due to the presence of remaining vaccine virus. After challenge, the amount of viral load detected in nasal swabs was significantly (P≤0.0001) lower T02 group compared to T01 group on Days 23, 25 and 28 corresponding to 2, 4- and 7-days post-challenge (See Table 7) and not significant on Day 31. The mean AUC of log viral load in nasal samples up to ten days post-challenge (from Day 21 to Day 31), as well as variation and ranges per each treatment are summarized in Table 8. The mean AUC value for the complete period was significantly (p<0.0001) lower in group T02 compared to T01 with mean values of 5.31±0.16 and of 7.03±0.10 respectively.

TABLE 7
Back-transformed Least Squares Mean PRRSV copies/ml in Nasal Swabs

| | D21 (before | | | | |
|---|---|---|---|---|---|
| Group | challenge) | D23 | D25 | D28 | D31 |
| T01 | 49.7 | 497429.7 | 2795257 | 21684.8 | 3394.3 |
| T02 | 305.6 | 10050.5 | 2719.6 | 552.3 | 1552.1 |
| P value | P < 0.0175 | P < 0.0001 | P < 0.0001 | P < 0.0001 | P = 0.3056 |

TABLE 8
Analysis of AUC PRRSV RNA copies/ml in
Nasal Swabs Following Challenge (Days > 21)

| Group | LSM | SE | Lower 95% CI | Upper 95% CI | Range |
|---|---|---|---|---|---|
| T01 | 7.03 | 0.10 | 6.83 | 7.22 | 6.35 to 7.8 |
| T02 | 5.31 | 0.16 | 4.98 | 5.64 | 3.8 to 6.65 |

P value
P < 0.0001

The presence of PRRS virus in Oral Swab samples was monitored by means of a RT-qPCR assay performed following local procedures. All pigs were found RT-qPCR PRRSV negative in oral swab samples before vaccination (Day<0).

On Day 21, before challenge, PRRSV RNA was detected in Oral Swab samples in 68.2% (15 out of 22) of the pigs in group T02 due to the presence of remaining vaccine virus. By that time, all T01 pigs remained PRRSV negative. On Day 23, two days post-challenge, 90.9% of piglets from T01 shed virus by nasal route reaching a peak four days post-challenge (Day 25) with 100% of the piglets from T01 shedding.

Frequencies of ever shedding by oral route during the monitored period showed shedding in 100% of the T02 piglets at one or more time points post-challenge (Days>21). However, percentages of animals T02 group with nasal shedding never reached 100% at a single time point.

Before challenge, on Day 21, the amount of viral load detected in Oral Swab samples was significantly (P<0.0001) higher in the pigs in T02 compared to the ones in T01 due to the presence of remaining vaccine virus. After challenge, the amount of viral load detected in Oral Swab samples was significantly (P≤0.05) lower in group T02 compared to control (T01) group on Days 25 and 28 corresponding to four- and seven-days post-challenge (See Table 9) and not significant on Days 23 and 31. The mean AUC of log viral load in oral samples up to ten days post-challenge (from Day 21 to Day 31), as well as variation and ranges per each treatment are summarized in Table 10. Differences on the mean AUC value for the complete period were not statistically significant (P=0.0690).

TABLE 9
Back-transformed Least Squares Mean PRRSV copies/ml in Oral Swabs

| | D21 (before | | | | |
|---|---|---|---|---|---|
| Group | challenge) | D23 | D25 | D28 | D31 |
| T01 | 50.0 | 12913.4 | 147949.3 | 16716.8 | 8776.0 |
| T02 | 6855.0 | 30940.0 | 18966.5 | 1924.3 | 2320.4 |
| P value | P < 0.0001 | P < 0.2013 | P < 0.003 | P < 0.0018 | P = 0.0542 |

TABLE 10
Analysis of AUC PRRSV RNA copies/ml in
Oral Swabs Following Challenge (Days > 21)

| Group | LSM | SE | Lower 95% CI | Upper 95% CI | Range |
|---|---|---|---|---|---|
| T01 | 5.85 | 0.14 | 5.57 | 6.13 | 4.78 to 7.53 |
| T02 | 5.49 | 0.14 | 5.21 | 5.77 | 3.74 to 6.67 |

P value
P < 0.069

### Rectal Temperatures

Differences of rectal temperature least square means between T01 and T02 in all monitored days post-challenge were not significant (not shown).

### Body Weights

Body weight means were significantly (P≤0.05) higher in T02 group compared to the control (T01) group before challenge (Day 21) and at the end of the study, ten days post-challenge (Day 31). Least Squares Means of body weight (kg) by treatment and time point are summarized in Table 11. Mean average daily weight gain (ADG) from the day of challenge (Day 21) until the end of the study (ten days post-challenge, Day 31), was of 0.11 kg/day for T01 piglets and 0.15 kg/day for T02 piglets. The difference in ADG between T01 and T02 (0.04 kg/day) was statistically significant (P=0.0245). Results of ADG are summarized in Table 12.

TABLE 11
Analysis of body weight-Least Squares Means
by Treatment and Time point (Kg)

| | Time point | | |
|---|---|---|---|
| | | D21 (pre- | |
| Group | D0 | challenge) | D31 |
| T01 | 1.71 | 5.71 | 6.95 |
| T02 | 1.87 | 6.34 | 7.98 |
| P-value | NS | P = 0.048 | P = 0.0018 |

TABLE 12
Analysis of body weight-Average Daily Gain
by Treatment and Time period (Kg/day)

| Group | Day 21 to 31 |
|---|---|
| T01 | 0.11 |
| T02 | 0.15 |
| P-value | P = 0.0245 |

### Serology

After birth and before vaccination on Day 0 all piglets were seronegative (S/P<0.4). Prior to challenge (Day 21) 100% of piglets from the control (T01) group remained seronegative (S/P<0.4) to PRRSV whereas 100% of piglets from T02 tested positive to PRRSV specific antibodies. At the end of the study, ten days post-challenge (Day 31), all piglets from T02 seroconverted and 100% of the pigs from T02 remained seropositive to PRRSV (data not shown).

The mean level of PRRSV specific antibodies was <0.4 the control (T01) group before challenge (Day 21) indicative of seronegativity whereas it was of 1.719 for T02 indicating seropositivity (0.4) of the group. Differences in mean S/P ratio were statically significative (P<0.0001) between treatment groups. At the end of the study, ten days post-challenge (Day 31), the mean level of PRRSV specific antibodies were >0.4 in both treatment groups indicating seropositivity in both T01 and T02. Nevertheless, the mean level of PRRSV specific antibodies was statistically (P<0.0001) higher T02 than in T01. See Table 13.

TABLE 13
Summary of Serology Least Square
Means (LSM) by group and day of study

| | D21 (before | D31 |
|---|---|---|
| Group | challenge) | |
| T01 | −0.001 | 1.412 |
| T02 | 1.719 | 2.031 |
| P-value | P < 0.0001 | P < 0.0001 |

### Example 2. Assessment of the Potential Effect of Maternally Derived Antibodies on the Efficacy of the SUVAXYN® PRRS MLV Administered to 3-Day-Old Seropositive Pigs by Intranasal Route

The objective of this study was to assess the efficacy of Suvaxyn® PRRS MLV vaccine in the presence of maternally derived antibodies (MDA) when administered to three-day-old seropositive piglets by intranasal (IN) route against challenge with a European strain of PRRSV-1 (Olot/91 strain).

Animal husbandry was similar to that described in Example 1, except sixty piglets were enrolled in the study 2-4 day old piglets were used for the study (fewer than 20% of 4-day old animals) and the piglets were Seropositive against PRRS. Presence of specific antibodies against PRRSV before vaccination (MDA+condition) was determined by means of an ELISA test as well as a seroneutralisation assay.

Similarly to the setup in Example 1, the piglets in group T01 were administered saline (1 ml per nostril) and the piglets in group T02 were administered SUVAXIN® PRRS MLV (1 ml per nostril, 10^{2.2} TCID₅₀ per 2 ml) on day 0. On Day 69 (when maternally-derived antibodies were undetectable), the piglets were challenged with PRRS-1 strain Olot/91 (1 ml per nostril, 10^{5.0} TCID50/2 mL.

Viremia and shedding, lung lesions, lung lesion scores, body weight, rectal temperatures and serology were determined and analyzed using the same procedures as described in Example 1.

### Results

### Viremia

The presence of PRRSV in serum was monitored by means of a RT-qPCR assay performed following local procedures. All pigs were found RT-qPCR PRRSV negative (if 550 PRRSV RNA copies/mL) in serum before vaccination (Day<0).

On Day 69, before challenge, PRRSV RNA was detected in 76.7% (23 out of 30) of the pigs in group T02. By that time, all T01 pigs remained PRRSV negative. By Day 71, 2 days post-challenge, 82.1% (23 out of 28) of piglets from T01 developed viremia reaching a peak 5 days post-challenge (Day 74) when 96.4% of T01 piglets were positive. See Table 14. Frequencies of ever viremic at any point throughout the monitored period confirmed that 100% of T01 developed viremia after challenge. Nevertheless, frequencies of viremic animals in T02 group never exceeded the 76.7%, and one animal remained PRRSV negative during the monitored period (data not shown).

TABLE 14
Percent of animals viremic for each treatment at each day

| | | D 69 pre- | | | | |
|---|---|---|---|---|---|---|
| Group | D < 0 | challenge | D 71 | D 74 | D 76 | D 78 |
| T01 | 0 | 0 | 82.1 | 96.4 | 78.6 | 82.1 |
| T02 | 0 | 76.7 | 71.9 | 53.1 | 59.4 | 68.8 |

Before challenge, on Day 69, the amount of viral load was significantly (P≤0.0001) higher in the T02 group compared to T01 due to the presence of remaining vaccine virus. After challenge, the amount of viral load detected in serum was significantly (P≤0.05) lower in the T02 group compared to T01 control group on Day 74 and 76 corresponding to 5 and 7 days post-challenge (See Table 15) and not significant on Days 71 and 78.

The mean Area Under the Curve (AUC) of log viremia up to 9 days post-challenge (from Day 69 to Day 78), as well as variation and ranges per each treatment are summarized in Table 16. PRRSV viremia as the mean AUC value for the complete period was significantly (P≤0.0001) lower in T02 group compared to T01 control group with mean values of 5.69±0.20 and of 6.87±0.16 respectively.

TABLE 15
Back-transformed Least Squares Mean
PRRSV RNA copies/mL in Serum

| | D69 pre | | | | |
|---|---|---|---|---|---|
| Group | challenge | D71 | D74 | D76 | D78 |
| T01 | 54 | 7372.9 | 1133183 | 120229.3 | 41818.2 |
| T02 | 3365.7 | 1678.1 | 585.3 | 4365.5 | 7218.9 |
| P value | P < 0.0001 | P = 0.0612 | P < 0.0001 | P = 0.0047 | P = 0.0825 |

TABLE 16
Analysis of AUC PRRSV RNA copies/mL
in Serum Post-Challenge (Days > 69)

| | | | Lower | Upper | |
|---|---|---|---|---|---|
| Group | LSM | SE | 95% CI | 95% CI | Range |
| T01 | 6.87 | 0.16 | 6.55 | 7.19 | 5.57 to 8.62 |
| T02 | 5.69 | 0.20 | 5.30 | 6.08 | 3.41 to 7.54 |
| P-value | P < 0.0001 | | | | |

### Lung Lesions

The percentage of lung with lesions for each treatment group is shown in Table 17. Comparison between treatment groups showed significant differences in the percentage of total lung with lesions (P=0.0175). The percentage of total lung with lesions was lower in T02 group compared to T01 group.

TABLE 17
Analysis of Percentage (%) of total lung with lesions

| | | Back Transform | | Lower | Upper | |
|---|---|---|---|---|---|---|
| Group | N | LSM | SE | 95% CI | 95% CI | Range |
| T01 | 28 | 3.42 | 0.913 | 1.83 | 5.49 | 0 to 25.50 |
| T02 | 32 | 1.36 | 0.416 | 0.65 | 2.32 | 0 to 10.20 |

| | |
|---|---|
| P value | P = 0.0175 |

Lung visual score by treatment is summarized in Table 18. At necropsy, 64.3% (18 out of 28) of piglets from T01 control group, had a positive lung visual score (score>0), indicating that PRRSV challenge was successful in inducing lung lesions. The percentage of animals scoring positive (score>0) in T02 was of 34.4% (11 out of 32) and significantly lower (P=0.0265)) than T01 group with 64.3% (18 out of 28) scoring positive. Even more, none of the animals vaccinated with the T02 scored 2 (Moderate lesions) whereas 10.7% (3 out of 28) animals from T01 did.

TABLE 18
Lung Visual Score by treatment group

| | Visual Score | | | | | | |
|---|---|---|---|---|---|---|---|
| | 0 | | 1 | | 2 | | Total |
| Group | N | % | N | % | N | % | observations |
| T01 | 10 | 35.7 | 15 | 53.6 | 3 | 10.7 | 28 |
| T02 | 21 | 65.6 | 11 | 34.4 | 0 | 0 | 32 |

| | |
|---|---|
| P-value | P = 0.0265 |

### Shedding

All pigs were found RT-qPCR PRRSV negative (if 550 PRRSV RNA copies/mL) in nasal swab samples before vaccination (Day<0). On Day 69, before challenge, PRRSV RNA was detected in nasal swab samples in 18.8% (6 out of 32) of the pigs group T02. By that time, all T01 pigs remained PRRSV negative. By Day 71, 2 days post-challenge, 7.1% (2 out of 28) of piglets from T01 shed virus by nasal route reaching a peak 5 days post-challenge (Day 74) when 96.4% (27 out of 28) of piglets vaccinated with saline (T01 group) were positive for shedding. Frequencies of ever shedding by nasal route during the monitored period confirmed that 100% of piglets in group T01 shed virus at one or more time points post-challenge (Days>69). Nevertheless, frequencies of animals in group T02 with nasal shedding never exceeded 56.3% (See Table 19) of the animals and, 9.4% of the animals remained negative for shedding during the monitored period.

TABLE 19
Percent of animals with nasal shedding
for each treatment at each day

| Group | D 0 | D 69 pre challenge | D 71 | D 74 | D 76 | D 78 |
|---|---|---|---|---|---|---|
| T01 | 0 | 0 | 7.1 | 96.4 | 96.4 | 46.4 |
| T02 | 0 | 18.8 | 43.8 | 37.5 | 56.3 | 50 |

Before challenge, some animals in T02 were positive due to the presence of vaccine virus, however differences in the amount of viral load detected in nasal swab samples were not significant. After challenge, the amount of viral load detected in nasal swabs was maintained significantly (P=0.0018) higher in the pigs in T02 group compared to piglets vaccinated with saline (T01 group) on Day 71, corresponding to two days post-challenge. However, the amount of viral load detected in nasal swab samples was significantly (P≤0.0001) lower in T02 compared to T01 on Days 74 and 76 corresponding to 5- and 7-days post-challenge (See Table 20) and not significant on Day 78.

The mean AUC of log viral load in nasal samples up to 9 days post-challenge (from Day 69 to Day 78), as well as variation and ranges per each treatment are summarized in Table 21. The mean AUC value for the complete period was significantly (P≤0.0001) lower in group T02 compared to T01 with mean values of 4.89±0.23 and of 6.53±0.10 respectively.

TABLE 20
Back-transformed Least Squares Mean
PRRSV copies/mL in Nasal Swabs

| | D69 pre | | | | |
|---|---|---|---|---|---|
| Group | challenge | D71 | D74 | D76 | D78 |
| T01 | 54.1 | 71.0 | 333749 | 380652.4 | 601.5 |
| T02 | 93.9 | 467.8 | 344.8 | 1754.1 | 1535.5 |
| P value | p = 0.0578 | P = 0.0018 | P < 0.0001 | P < 0.0001 | P = 0.2852 |

TABLE 21
Analysis of AUC PRRSV RNA copies/ml in
Nasal Swabs Following Challenge (Days > 69)

| | | | Lower | Upper | |
|---|---|---|---|---|---|
| Group | LSM | SE | 95% CI | 95% CI | Range |
| T01 | 6.53 | 0.10 | 6.33 | 6.72 | 5.61 to 7.33 |
| T02 | 4.89 | 0.23 | 4.43 | 5.36 | 2.65 to 6.89 |
| P value | P < 0.0001 | | | | |

The presence of PRRSV in oral swab samples was monitored by means of a RT-qPCR assay performed following local procedures. All pigs were found RT-qPCR PRRSV negative (if ≤50 PRRSV RNA copies/mL) in oral swab samples before vaccination (Day<0). On Day 69, before challenge, PRRSV RNA was detected in oral swab samples in 21.9% (seven out of 32) of the pigs in T02. By that time, all T01 pigs remained PRRSV negative. By Day 71, 2 days post-challenge, 10.7% of piglets from T01 shed virus by oral route reaching a peak five days post-challenge (Day 74) when 89.3% of piglets vaccinated with saline (T01 group) were positive shedding. Frequencies of ever shedding by oral route during the monitored period confirmed that 100% of piglets vaccinated with saline (T01 group) shed virus at one or more time points post-challenge (Days>69). Nevertheless, frequencies of animals with oral shedding within group T02 never exceeded the 68.8% (See Table 22) of the animals and, 9.4% of the animals maintained shedding negative during the monitored period. Frequencies of oral shedding animals, per treatment group at each time-point are detailed in Table 22.

TABLE 22
Percent of animals with nasal shedding
for each treatment at each day

| Group | D 0 | D 69 pre challenge | D 71 | D 74 | D 76 | D 78 |
|---|---|---|---|---|---|---|
| T01 | 0 | 0 | 10.7 | 89.3 | 82.1 | 85.7 |
| T02 | 0 | 21.9 | 36.7 | 50.0 | 37.5 | 68.8 |

Before challenge, on Day 69, the amount of viral load detected in Oral Swab samples was significantly (P=0.0085) higher in the pigs in group T02 compared to the ones vaccinated with saline (T01 group) due to the presence of remaining vaccine virus. After challenge, the amount of viral load detected in oral swabs was maintained significantly (P 0.05) higher level in the pigs in T02 compared to piglets in T01 on Day 71, corresponding to two days post-challenge. However, the amount of viral load detected in Oral Swab samples was significantly (P≤0.05) lower in T02 group compared to T01 control group on Days 74 and 76 corresponding to 5- and 7-days post-challenge (See Table 23) and not significant on Day 78. The mean AUC of log viral load in oral samples up to nine days post-challenge (from Day 69 to Day 78), as well as variation and ranges per each treatment are summarized in Table 24. The mean AUC value for the complete period was significantly (P=0.0212) lower in T02 piglets compared to T01 piglets with mean values of 4.33±0.16 and of 4.81±0.12 respectively.

TABLE 23
Back-transformed Least Squares Mean
PRRSV RNA copies/ml in Nasal Swab

| | D69 pre | | | | |
|---|---|---|---|---|---|
| Group | challenge | D71 | D74 | D76 | D78 |
| T01 | 52.4 | 78.8 | 5372.9 | 1803.8 | 3928.0 |
| T02 | 105.6 | 211.5 | 351.4 | 308.7 | 2778.6 |
| P value | p = 0.0085 | p = 0.0308 | P < 0.0001 | P = 0.0077 | P = 0.6287 |

TABLE 24
Analysis of AUC PRRSV RNA copies/mL in
Oral Swabs Following Challenge (Days > 69)

| | | | Lower | Upper | |
|---|---|---|---|---|---|
| Group | LSM | SE | 95% CI | 95% CI | Range |
| T01 | 4.81 | 0.12 | 4.56 | 5.06 | 3.74 to 6.14 |
| T02 | 4.33 | 0.16 | 4.02 | 4.65 | 2.65 to 6.2 |
| P value | P = 0.0212 | | | | |

### Rectal Temperatures

Before challenge, on Day 69, a significantly higher (P<0.0001) rectal temperature was evidenced in group T02 compared to T01 with least square means of 40.0 and 39.5° C. respectively. However, as none of the animals had fever, means were within the expected range for the variable and considering that animals had been just comingled, it was considered not relevant. The least squares mean rectal temperature was significantly (P=0.0007) higher in T01 group compared to T02 group piglets at Day 74 however none of the animals from both treatment groups showed fever that day. On the other monitored days post-challenge, differences in rectal temperature between groups were not significant.

### Body Weight

Comparison of least square means between the groups showed no significant differences (P>0.05) in body weight before challenge (Day 69) as well as at the end of the study 9 days post-challenge (Day 78). Nevertheless, mean ADG from day of challenge (Day 69) until the end of the study nine days post-challenge (Day 78) was of 0.69 Kg/day for T01 piglets and 0.56 Kg/day for T02 piglets. The difference in ADG between T01 and T02 (0.14 Kg/day) was statistically significant (P=0.0226). Results of ADG are summarized in Table 25.

TABLE 25
Analysis of Body Weight-Average Daily Gain
by Treatment and Time period (Kg/day)

| Group | Day 69 to 78 |
|---|---|
| T01 | 0.69 |
| T02 | 0.56 |
| P value | P = 0.0226 |

### Serology

After birth, on Day<0 (<3 days of age) antibodies specific to PRRSV were detected by ELISA in 90.9% (30 out of 33) of piglets from group T01 and in 96.9% (31 out of 32) of piglets from group T02 (See Table 26). As per protocol all piglets should be seropositive to PRRSV (MDA+) to be enrolled before vaccination. The four piglets, in which antibodies specific to PRRSV were not detected by ELISA test, were verified to be serologically positive (seroneutralizing titre>2) to PRRSV by means of the seroneutralization test.

Prior to challenge (Day 69) 92.9% (26 out of 28) of piglets from T01 control group tested negative (S/P<0.4) to PRRSV by means of ELISA test. Of the piglets from group T02, only 3.3% (1 out of 30) of the animals tested negative to PRRSV specific antibodies. At necropsy (Day 78), 64.3% (18 out of 28) of piglets from T01 control group seroconverted while 100% of the pigs from T02 group were seropositive to PRRSV.

Serology least squares means for each treatment at each time point are summarized in Table 27. The mean level of PRRSV specific antibodies was <0.4 for group T01 before challenge (Day 69) indicative of seronegativity whereas it was of 1.880 for group T02 indicating seropositivity (>0.4) of the group. Differences in mean S/P ratio were statically significative (P≤0.0001) between treatment groups. At the end of the study (Day 78=9 days post-challenge), the mean level of PRRSV specific antibodies were >0.4 in both treatment groups indicating seropositivity in T01 as well as in T02. Nevertheless, mean level of PRRSV specific antibodies was statistically higher in T02 than in T01.

TABLE 26
Percent Distribution for Seroconversion (S/P Ratio ≥
0.4) for Each Treatment at Each Time Point

| | | Seroconversion | | | | |
|---|---|---|---|---|---|---|
| | Study | NO | | YES | | Observations, |
| Treatment | Day | N | % | N | % | N |
| T01 | D < 0 | 3 | 9.1 | 30 | 90.9 | 33 |
| | D 69 | 26 | 92.9 | 2 | 7.1 | 28 |
| | D 78 | 10 | 35.7 | 18 | 64.3 | 28 |
| T02 | D < 0 | 1 | 3.1 | 31 | 96.9 | 32 |
| | D 69 | 1 | 3.3 | 29 | 96.7 | 30 |
| | D 78 | 0 | 0 | 32 | 100 | 32 |

TABLE 27
Summary of Serology Least Square Means
(LSM) by group and day of study

| Group | D69 pre-challenge | D78 |
|---|---|---|
| T01 | 0.156 | 0.767 |
| T02 | 1.880 | 1.840 |
| P value | P < 0.0001 | P < 0.0001 |

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of inducing protective immunity against Porcine Reproductive and Respiratory Virus type 1 (PRRS-1) in a piglet that is older than 60 hours of age, the method comprising intranasally administering to said piglet a vaccine comprising a modified live PRRS-1 virus attenuated in cells expressing porcine CD163, wherein said piglet is PRRS-1 MDA-positive.

2. The method of claim 1, wherein the genome of said modified live PRRS-1 virus comprises an RNA molecule that is SEQ ID NO: 1 or that is at least 75% identical to SEQ ID NO: 1, and wherein further,
a) the amino acid sequence encoded by ORF1a of the genome of the modified live PRRS-1 virus in said vaccine contains:
S, A, or T, preferably S, at amino acid position 19;
Y F, or W, preferably Y, at amino acid position 157;
D or E, preferably D, at amino acid position 268;
H, R, or K, preferably H, at amino acid position 294;
Y, F, or W, preferably Y, at amino acid position 416;
S, A, or T, preferably S, at amino acid position 742;
L, I, M, or V, preferably L, at amino acid position 884;
P at amino acid position 908;
K, R, or H, preferably K, at amino acid position 916;
K, R, or H, preferably K, at amino acid position 977;
S, A, or T, preferably S, at amino acid position 1138;
F, Y, or W, preferably F, at amino acid position 1160;
S, A, or T, preferably S, at amino acid position 1500;
R, K, or H, preferably R, at amino acid position 2094;
P at amino acid position 2254; and
L, I, M, or V, preferably L, at amino acid position 2290; and
b) the amino acid sequence encoded by ORF1b of the genome of the modified live PRRS-1 virus in said vaccine contains:
S, A, or T, preferably S, at amino acid position 567; and
H, R, or K, preferably H, at amino acid position 912; and
c) the amino acid sequence encoded by ORF2a of the genome of the modified live PRRS-1 virus in said vaccine contains:
L, I, M, or V, preferably L, at amino acid position 22;
F, Y, W, preferably F, at amino acid position 88;
M, L, I, or V, preferably M, at amino acid position 94; and
F, Y, or W, preferably F, at amino acid position 95; and
d) the amino acid sequence encoded by ORF2b of the genome of the modified live PRRS-1 virus in said vaccine contains:
L, I, M, or V, preferably L, at amino acid position 47; and
e) the amino acid sequence encoded by ORF4 of the genome of the modified live PRRS-1 virus in said vaccine contains:
T, S, or A, preferably T, at amino acid position 151 and
f) the amino acid sequence encoded by ORF5 of the genome of the modified live PRRS-1 virus in said vaccine contains:
F, Y, or W, preferably F, at amino acid position 20; and
D or E, preferably D, at amino acid position 37; and
g) the amino acid sequence encoded by ORF5a of the genome of the modified live PRRS-1 virus in said vaccine contains:
V, L, I, or M, preferably V, at amino acid position 18; and
R, H, or K, preferably R, at amino acid position 35.

3. The method of claim 2, wherein
a) the amino acid sequence encoded by ORF1a of the genome of the modified live PRRS-1 virus in said vaccine further contains Y at amino acid position 24; and A at amino acid position 156; and
b) the amino acid sequence encoded by ORF3 of the genome of the modified live PRRS-1 virus in said vaccine further contains S at amino acid position 52.

4. The method of claim 3, wherein the genome of said modified live PRRS-1 virus comprises an RNA molecule that is at least 90% identical to SEQ ID NO: 1.

5. The method of claim 1, wherein the genome of said modified live PRRS-1 virus comprises an RNA molecule that is at least 90% identical to SEQ ID NO: 1.

6. The method of claim 1, wherein the genome of said modified live PRRS-1 virus comprises an RNA molecule that is at least 95% identical to SEQ ID NO: 1.

7. The method of claim 2, wherein at least 50% of the differing nucleotides between the genome of said modified live PRRS-1 virus and SEQ ID NO: 1 result in
a) silent mutations or
b) conservative substitutions or
c) a combination thereof.

8. The method of claim 1, wherein said piglet is at least 64 hours old, or at least 68 hours old or at least 72 hours old, or at least 76 hours old, or at least 80 hours old or at least 84 hours old.

9.-13. (canceled)

14. The method of claim 1, wherein the piglet is 28 days of age or younger, or 21 days of age or younger, or 14 days of age or younger, or 10 days of age or younger, 7 days of age or younger, 5 days of age or younger.

15.-19. (canceled)

20. The method of claim 1, wherein said protective immunity comprises at least one of reduced viremia, reduced viral shedding, lung lesion scoring, and lung lesion frequency.

21.-23. (canceled)

24. The method of claim 1, wherein said protective response further comprises weight gain compared to unvaccinated infected piglets.

25. The method of claim 1, wherein said vaccine is a single-dose vaccine.

26. The method of claim 1, wherein an effective dose of said modified live PRRS-1 virus in the vaccine is at least 10^{2.0} TCID₅₀.

27. The method of claim 25, wherein an effective dose of said modified live PRRS-1 virus in the vaccine is about 10^{2.2} TCID₅₀ to about 10^{6.0} TCID₅₀, or about 10^{2.2} TCID₅₀ to about 10^{5.2} TCID₅₀.

28. (canceled)

29. The method of claim 25, wherein an effective dose of said modified live PRRS-1 virus in the vaccine is about 10^{2.2} TCID₅₀, or about 10^{2.5} TCID₅₀, or about 10^{2.8} TCID₅₀, or about 10^{3.1} TCID₅₀, or about 10^{3.4} TCID₅₀, or about 10^{3.7} TCID₅₀, or about 10^{4.0} TCID₅₀, or about 10^{4.4} TCID₅₀, or about 10^{4.8} TCID₅₀, or about 10^{5.2} TCID₅₀, or about 10^{5.6} TCID₅₀.

30. The method of claim 1, wherein the volume of said vaccine administered to the piglet is from about 0.5 ml to about 5 ml.

31. The method of claim 1, wherein the volume of said vaccine administered to the piglet is about 2 ml.
